(19) United States
(12) Patent Application Publication
(10) Pub. No.: US 2004/0115742 A1
(43) Pub. Date: Jun. 17, 2004

(54) Method to identify specific interaction between ligand and receptor

(76) Inventors: Ruoying Tan, Foster City, CA (US); Xiao-Hong Qian, San Jose, CA (US); Yibing Li, San Francisco, CA (US)

Correspondence Address: Cameron A. King, Morrison & Foerster LLP, 425 Market Street, San Francisco, CA, 94105-2482, US

(21) Appl. No.: 10/609,192

(22) Filed: Jun. 27, 2003

Related U.S. Application Data
(60) Provisional application No. 60/392,884, filed on Jun. 28, 2002.
(60) Provisional application No. 60/400,627, filed on Aug. 2, 2002.

(51) Int. Cl. C12Q 1/68; G01N 33/53; G01N 33/567

(52) U.S. Cl. 435/7.2; 435/6

(57) ABSTRACT

The invention provides methods for quickly and efficiently detecting receptor-ligand binding, including high throughput, cell-based assay methods. Assay methods for detecting mediators of receptor-ligand binding and for screening cDNA libraries are also provided.

This application claims the benefit of U.S. Provisional Applications No. 60/392,884, filed Jun. 28, 2002, and Ser. No. 60/400,627, filed Aug. 2, 2002, both are incorporated herein by reference.

### FIELD OF THE INVENTION

This invention relates to cell-based assays for identifying interactions between ligand-receptor pairs. Specifically, methods for screening known receptors for ligands that bind to the receptors and known ligands for receptors that bind to the ligands are described. Methods for screening mediators of ligand-receptor binding are also provided.

### BACKGROUND OF THE INVENTION

Communication between cells and their environment is crucial for cellular growth, death and differentiation. The key molecules responsible for the communication are ligands and receptors. The information is transmitted by ligands and is in turn received and interpreted by a diverse set of cell surface receptors to which the ligands bind.

Ligands generally consist of two broad categories: small molecules and large molecules. Small molecular ligands include biogenic amines, amino acids, ions, lipids, nucleotides, and chemical compounds that represent the majority of classic drugs. Most large molecular ligands are secreted polypeptides (i.e., secreted proteins or smaller peptides), including antibodies, growth factors, interleukins, cytokines, and some enzymes, etc. Cell surface receptors, which are membrane proteins, represent most targets of drugs currently being marketed.

Since the decoding of genomes of human and other organisms, a large number of secreted proteins and membrane proteins have been identified or predicted, and many of these proteins are considered novel ligands or orphan receptors. Elucidation of the function of these proteins and identification of specific ligand-receptor pairs present both challenges and rewards in the post-genomic era.

Ligands and receptors are regarded as attractive targets for drug development. Many marketed drugs function in mediating (that is, inhibiting or enhancing) the interaction between ligands and their receptors. There are currently certain assays that have been attempted to identify specific interactions between ligands and receptors; and yet other assays have been attempted to screen small molecule compounds that can specifically mediate (inhibit or enhance) the interactions between ligands and their receptors. However, many such assays require time-consuming procedures including purifying and labeling ligands or receptors, and none provide a highly sensitive, high throughput cell-based assay capable of quickly and efficiently screening large numbers of ligands or receptors from, e.g., an entire cDNA library.

Many enzymes, such as beta-lactamase, alkaline phosphatase, leucificerase, and beta-galactosidase have been used as genetic reporters to monitor biological events. For example, use of a leaderless beta-lactamase gene or a leaderless alkaline phosphatase as a reporter to identify signal peptides sequences have been reported (see Moore, et al., (1997) Annal. Biochem. 247, 203; Chubb, A. J. et al. (1998) Microbiology 144, 1619; Chen, H. and Leder, P. (1999) Nucleic Acids Research, 27, 1219, and U.S. Pat. Nos. 5,801,000, and 5,554,499). Also, the use of beta-lactamase fluorescent substrate CCF2 (Aurora) allows for quantitation of gene expression and clonal selection of single living cells (see Zlokamik, G. et al., (1998) Science 279, 84; and Raz, E. et al., (1998) Development Biology 203 290).

However, issues remain in these currently available methods that hinder their application in drug development. For example, some procedure requires a heat inactivation step in order to reduce the background noise during the experimental data gathering stage, as in the case of alkaline phosphatase. Therefore, it remains of considerable biomedical and pharmaceutical benefit to provide a highly sensitive, high throughput cell-based assay system for identifying specific interactions between ligands and receptors, methods for screening polypeptide ligands for their corresponding receptors, and methods for finding small molecule drugs that can mediate the ligand-receptor binding process.

### SUMMARY OF THE INVENTION

The present invention provides an efficient, and highly sensitive approach to identify specific interactions between ligands and receptors. The present invention also provides methods for screening large number of polypeptide molecules for receptor-ligand binding activity in a high throughput manner, including methods amenable to cell-based assay systems.

According to one aspect of the invention, a method is provided for identifying, in a sample, a receptor which is capable of binding to a known ligand, including providing a fusion molecule comprising the known ligand covalently linked to a threshold reporter enzyme molecule, the threshold reporter enzyme molecule being capable of reacting with a suitable substrate so as to generate a detection signal, contacting the sample containing the receptor with the fusion molecule to form a complex between the receptor and the known ligand, and detecting the presence of the complex by incubating the complex with the substrate so as to generate a detection signal indicative of receptor-ligand binding.

According to another aspect of the invention, a method is provided for identifying a ligand from a plurality of polypeptide molecules in a sample, said ligand being capable of binding to a known receptor, including providing a fusion molecule comprising the ligand covalently linked to a threshold reporter enzyme molecule, the threshold reporter enzyme molecule being capable of reacting with a suitable substrate to generate a detection signal, contacting the sample containing the known receptor with the fusion molecule to form a complex between the known receptor and the ligand, and detecting the presence of the complex by incubating the complex with the substrate so as to generate a detection signal indicative of receptor-ligand binding.

In the various preferred embodiments of the invention, the threshold reporter enzyme in the fusion molecule can be beta-lactamase, and the suitable substrate can be CCF2, or nitrocefin. The ligand can be soluble, which is secreted, or insoluble, which is bound to a cell surface, or a virus. The ligand can be naturally occurring polypeptide, or recombinant polypeptide or polypeptide fragments. The receptor can be cell surface receptor. The sample can be a cell sample containing cell surface receptor bound to the membrane of the cells. The detection signal can be in the form of a fluorescent signal, chemiluminescent signal, or a colorimetric signal.

According to yet another aspect of the invention, a method is provided for identifying a compound that mediates the binding activity between a known ligand and a known receptor, including providing a fusion molecule comprising the known ligand covalently linked to a threshold reporter enzyme molecule, the threshold reporter enzyme molecule being capable of reacting with a suitable substrate so as to generate a detection signal, contacting the fusion molecule with the known receptor in the presence of the compound, so as to form a complex between the known receptor and the known ligand, detecting the presence of the conjugate by incubating the complex with the substrate so as to generate a detection signal, and determining the amount of the detection signal and comparing that amount to an amount of a detection signal obtained in the absence of the compound. A reduction in the amount of the detection signal in the presence of the compound indicates that the compound inhibits the binding of the ligand to the receptor. An increase in the amount of the detection signal in the presence of the compound indicates that the compound enhances the binding of the ligand to the receptor.

In various further preferred embodiments of the invention, the compound which mediates the ligand-receptor binding can be an organic chemical compound, or an inorganic chemical compound. The compound can also be a small peptide molecule such as EMP-1. The pair of ligand/receptor which can be used in screening for small compounds that mediate the binding can be the tumor necrosis factor alpha/tumor necrosis factor receptor 2, the interleukin-8/interleukin-8 receptor A, and erythropoietin/erythropoietin receptor pairs.

In yet another embodiment of the invention, a method for identifying a compound that blocks viral entry is provided wherein the ligand is a viral envelope protein, preferably the viral glycoprotein, or gp120, or gp41, and the receptor is cellular viral receptor protein, preferably CD-4.

In a further embodiment of the invention, a method for identifying a compound that blocks viral entry is provided wherein the receptor is a viral envelope protein, preferably the viral glycoprotein, or gp120, or gp41, and the ligand is cellular viral receptor protein, preferably CD-4.

According to another aspect of the invention, a composition is provided for use in identifying specific binding activity between a ligand and a receptor, comprising a ligand covalently linked to a threshold reporter enzyme molecule, the threshold reporter enzyme molecule being capable of reacting with a suitable substrate so as to generate a detection signal.

According to yet a further aspect of the invention, a composition is provided for use in identifying a compound which interferes with the binding of a known receptor to a known ligand comprising said known ligand covalently linked to a threshold reporter enzyme molecule, the threshold reporter enzyme molecule being capable of reacting with a suitable substrate so as to generate a detection signal.

### BRIEF DESCRIPTION OF THE DRAWINGS

FIG. 1A shows a construct according to one variation of the invention for expressing a ligand-lactamase fusion. A gene encoding polypeptide ligand is linked to the 5′ end of a leaderless beta-lactamase gene with a flexible linker, the whole fusion gene being under control of a promoter.

FIG. 1B shows a construct according to one variation of the invention for expressing receptor. The gene encoding the receptor is under the control of a eukaryotic (e.g., mammalian) promoter.

FIG. 2A illustrates the cell-based assay according to one variation of the invention for screening binding of ligand lactamase fusion proteins to a known receptor.

FIG. 2B illustrates the cell-based assay according to one variation of the invention for screening binding of receptors to a known ligand lactamase fusion protein.

FIG. 3 demonstrates the high fluorescent signal to noise ratio obtained with binding of ligand (EPO, IL-8, and TNF) to its specific receptor (EPOR, IL-8R, TNFR) using beta-lactamase as the reporter molecule and CCF2 as the substrate.

FIG. 4 shows the fluorescent signal to noise ratio obtained with binding of a specific receptor (EPOR, IL-8R, TNFR) to its corresponding ligand (EPO, IL-8, and TNF) in a multiplexed pool using beta-lactamase as the reporter molecule and CCF2 as the substrate.

FIG. 5 shows the fluorescent signal to noise ratio obtained with binding of a specific ligand (EPO, IL-8, and TNF) to its corresponding receptor (EPOR, IL-8R, TNFR) in a multiplexed pool using beta-lactamase as the reporter molecule and CCF2 as the substrate.

FIG. 6 shows the fluorescent readout obtained from binding of EPOR to EPO. The EPO ligand was screened from a cDNA expression library having a multiplexing factor of 12.

FIG. 7 shows the specific inhibition of binding of the EPO ligand to its receptor, EPOR by peptide EMP 1.

### DETAILED DESCRIPTION OF THE INVENTION

Definitions:

The term “Receptor” herein refers to a molecule, usually a cell surface protein, that has a binding site with high affinity for a particular signaling substance, such as a hormone, or a neurotransmitter.

The term “Ligand” herein refers to a molecule that binds to the Receptor, and initiates the signal transduction process in the cell. The ligand can be a polypeptide produced naturally by a living organism, or synthesized by man using fragments of polypeptide molecules.

The term “threshold reporter enzyme molecule” herein is defined as an enzyme molecule that is not found endogenously in cells expressing the receptor, or an enzyme that is capable of being detected at a concentration of at least 5 fM.

The term “Fusion molecule” herein refers to a recombinant polypeptide containing a ligand molecule and the reporter enzyme molecule.

The term “Detection signal” herein refers to an optical signal capable of giving out a readout when detected by optical instrumentation such as flow cytometry instruments. Examples of a detection signal include fluorescent signal, a chemiluminescent signal, or a colorimetric signal.

The term “suitable substrate” herein refers to a substrate for the reporter enzyme which can be catalyzed by the reporter enzyme, and converted into a different end product that generates a detection signal.

The term “Complex” herein refers to the affinity complex formed between a ligand and its associated receptor under appropriate binding conditions.

The term “Mammalian” herein refers to any mammalian species such as human, mouse, rat, and so forth.

The term “beta lactamase” herein refers not only to the intact enzyme molecule of beta-lactamase, but also to a polypeptide that contains the catalytic domain of beta-lactamase, and other functional derivatives of beta-lactamase.

Molecules which mediate or modulate receptor-ligand binding include molecules which are capable of either inhibiting the binding specificity of a known ligand to its known receptor, or increasing the binding specificity of a known ligand to its known receptor. Examples of a mediating or modulating molecule are small molecules such as organic compounds, inorganic compounds, small peptide molecules, peptide fragments, and so forth.

The methods of the invention include cell-based assays. By the term “cell-based assay” it is meant that a nucleic acid encoding a receptor is introduced into and expressed in a cell line, the cell line expressing the receptor contacts culture medium containing a ligand reporter fusion molecule; and binding of the ligand-reporter fusion molecule to the receptor is then detected by detecting the presence of the threshold reporter enzyme molecule.

Natural or recombinant polypeptide ligands may be screened for specific receptors from a pool of ligands. Here, the term “natural polypeptide ligand” means the polypeptide is encoded by a cDNA species. The term “recombinant polypeptide ligand” refers to a polypeptide ligand, e.g., from a pool of polypeptides, in which a region of the polypeptide is encoded by a pool of combinatorial nucleotides and where it is anticipated that some of polypeptide ligand species generated may offer a tighter binding to the specific receptor. The library expressing natural or recombinant polypeptide ligands is generated by fusing random primed reverse transcribed cDNAs or combinatorial polynucleotides to the 5′-end of a leaderless beta-lactamase gene (or other reporter gene). Expression of the fusion gene in an extracellular environment is confirmed by assays that detect the secretion of ligand fusion proteins (Chubb, A. J. et al. (1998) Microbiology 144, 1619; Chen, H. and Leder, P. (1999) Nucleic Acids Research, 27, 1219). Further high throughput screening of the library expressing natural or recombinant polypeptide fusions against cells expressing a specific receptor should detect natural or recombinant polypeptide ligands that bind to the specific receptor with higher binding affinity than that of a reference ligand. In the same manner, natural or recombinant receptors may be screened.

When a cDNA library is used, multiplexing allows the number of samples to be reduced, and decreases the time for assay completion. Because our cell-based assays, as further described, are able to detect receptor-ligand binding with as little as 5 fM of reporter molecule present, efficiency of the assay is also increased. Ligand and/or receptor samples may be multiplexed to include at least about 10, at least about 20 at least about 30, at least about 40, at least about 50, at least about 60, at least about 70, at least about 80, at least about 90, or at least about 100 ligands or receptors. For example, a cDNA library having about one million samples may be reduced to 100,000 after enrichment. By then using a multiplexing factor of 96 (i.e., 96 ligands or receptors in a sample), the number of samples can then be reduced to approximately 1000. As a result, the complete assay for screening one million samples (of ligand or receptor) may be conducted using about ten 96-well plates.

Reporter molecule and substrate systems suitable for use with our cell-based assays are ones that achieve the desired sensitivity to detect receptor-ligand binding in a sample. We have surprisingly found that beta-lactamase is one such suitable reporter molecule. Without being bound by theory, we believe that the required level of assay sensitivity is provided by reporter molecules, such as beta-lactamase, that are non-endogenous to mammalian cell lines and which are of a size that minimizes potential stearic interference or hindrance of the desired receptor-ligand binding. In one variation, beta-lactamase together with the fluorogenic substrate CCF2 or any of its derivatives may be used as a reporter-substrate system. Upon cleavage by beta-lactamase, a change in fluorescence emission from CCF2 is exhibited, and is detectable at a concentration of beta-lactamase of as little as 5 femtomolar (fM), allowing for highly sensitive detection of very small quantities of sample. Furthermore, beta-lactamase is not endogenous to most cell lines, and is a small molecule (about 700 bp), not prone to stearic hindrance. Again without being bound by theory, we believe the inapplicability or inadequacy of other assay systems compared to our high throughput, highly sensitive, cell based assay system may be attributed to use of reporter molecules that are endogenous to many cell types, and which then require heating of samples to eliminate the endogenous molecules (frequently killing the cells or denaturing proteins of interest); or use of large reporter molecules that are prone to stearic hindrance. These other systems do not provide a highly sensitive, high throughput cell-based assay capable of quickly and efficiently screening large numbers of ligands or receptors from, e.g., an entire cDNA library. Other reporter molecules and substrate systems are also envisaged, including, but not limited to, enzymes such as alkaline phosphatase, leuciferase, and beta-galactosidase, and other marker molecules such as green fluorescent protein.

The cell-based assays of this invention may generally be employed to detect cognate (known) receptors for orphan (unknown) ligands, cognate (known) ligands for orphan (unknown) receptors, and to screen for compounds that mediate receptor-ligand binding. Any receptor-ligand pair may be detected, so long as it may be expressed in a cell-based assay, as described above.

For example, the following polypeptide ligands and receptors have been expressed and detected using the inventive cell-based assays: tumor necrosis factor alpha (TNF, P01375) and tumor necrosis factor receptor 2 (TNFR, P20333) (Smith, C. A. et. al., (1990) Science 248, 1019); interleukin-8 (IL8, P10145) and interleukin-8 receptor A (IL8R, P25024) (Lee, J. et al. (1992) J. Biol. Chem. 267, 16283); erythropoietin (EPO, P01588) and erythropoietin receptor (EPOR, P19235) (D'Andrea. et al., (1989) Cell 57, 277); Constructs expressing the fusion genes of these polypeptide ligands with lactamase, and also constructs expressing their receptors have been generated.

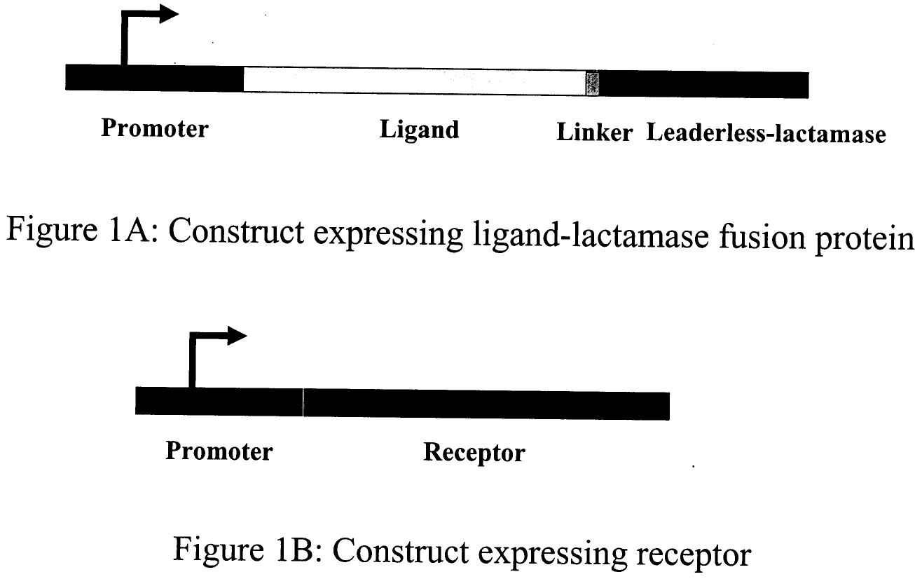

In a variation of the invention, the cell-based assay may be used to screen and identify an unknown receptor. In general, a DNA construct expressing polypeptide ligand is generated by fusing DNA sequences encoding the polypeptide ligand to the 5′ end of a leaderless beta-lactamase gene. The fusion gene is under the control of a promoter (FIG. 1A); the promoter may be a prokaryotic (e.g., bacterial) promoter or a eukaryotic (e.g., mammalian) promoter, or a dual expression promoter (functions both in prokaryotic system and eukaryotic system). The plasmid expressing the polypeptide ligand may contain a gene encoding, e.g., neomycin, for G418 selection (Invitrogen).

A cDNA expression library may also be generated by ligating cDNAs into a eukaryotic (e. g., mammalian) expression vector as described (SUPERSCRIPT™ Plasmid System for cDNA Synthesis and Plasmid Cloning (GIBCOBRL, CAT.NO 212220), and as depicted in Example 5. The cDNAs are prepared by an oligo dT primed method (SUPERSCRIPT™ Plasmid System for cDNA Synthesis and Plasmid Cloning (GIBCOBRL, CAT.NO 212220). The vector for constructing the cDNA library may contain a gene encoding, e.g., neomycin, for G418 selection (Invitrogen). The ligation products are then transformed into bacterial cells. The resulting colonies are picked into multiple well plates to grow. The plasmid DNA is prepared using Quick kit (Qiagen), and transfected into eukaryotic (e.g., mammalian) cells in a multiple-well format. The plasmid expressing a ligand lactamase fusion gene is transfected into eukaryotic (e.g., mammalian) cells or is transformed into prokaryotic (e.g., bacterial) cells. A cDNA expression library for receptors may also be generated using this method, further exemplified in Example 2.

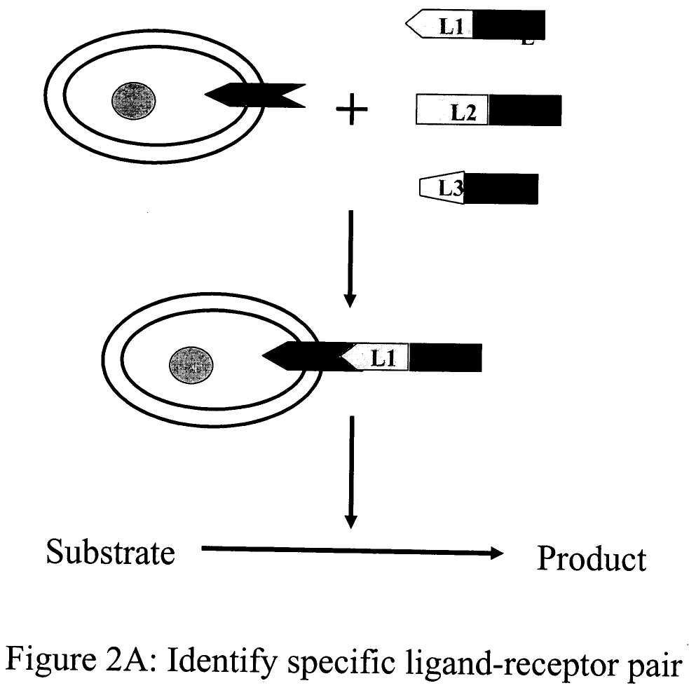

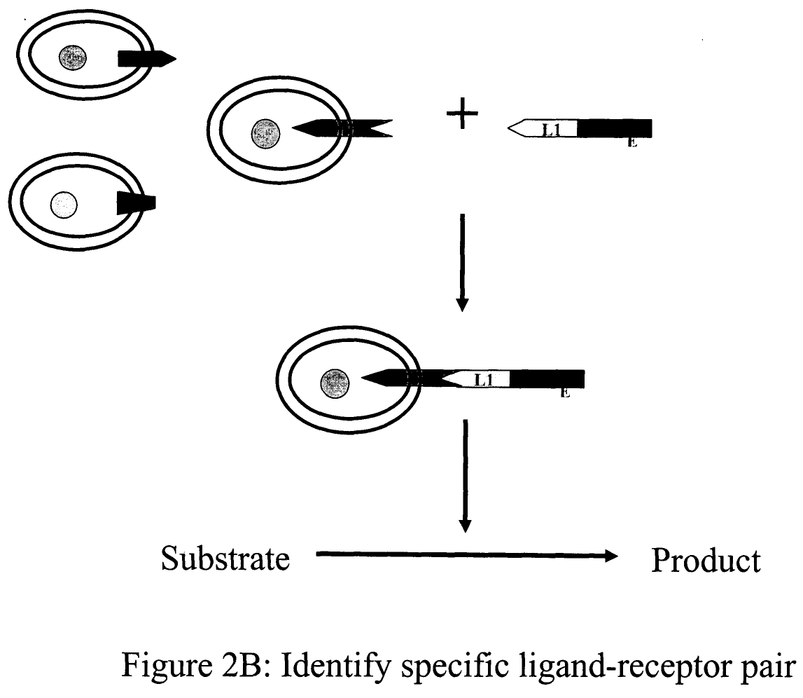

Cells transfected with plasmids of the cDNA library contact the culture medium of cells expressing the polypeptide-ligand fusion. If cells in some well(s) transfected with plasmids of the cDNA library express receptor for the specific polypeptide ligand, the polypeptide ligand will bind to the surface of cells in some well(s), and cannot be washed off. As seen in FIG. 2A, the ligand-fusion bound on the cell surface cleaves added lactamase substrate (e.g., CCF2 (Aurora)), and displays a change in fluorescence emission in this (or these) well(s). The plasmids transfected into the well(s) will then be sequenced. The gene encoding specific receptor for the known specific ligand is thereby identified.

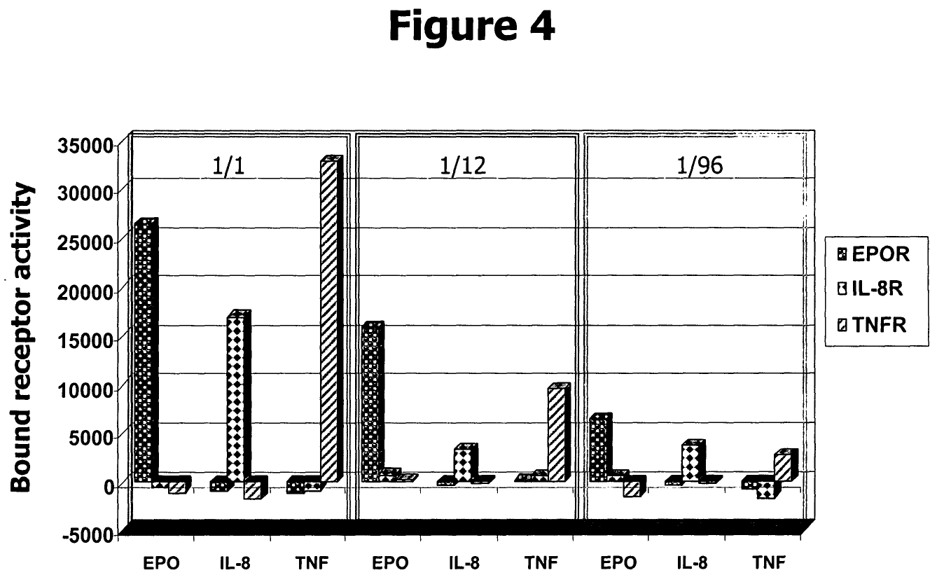

The unknown receptor may also be identified from a multiplexed pool of ligands using, e.g., a beta-lactamase reporter and CCF2 as the substrate. As shown in FIG. 4, the EPO, IL-8, and TNF ligands were placed in a pool of both 12 and 96 ligands. Compared with the fluorescent readouts (in arbitrary units) for one to one binding of receptor and ligand (26398 for EPO/EPOR; 16941 for IL-9/IL-8R; and 32899 for TNF/TNFR), binding of specific receptor-ligand pairs from a multiplexed pool of 12 ligands (13570 for EPO/EPOR; 3785 for IL-8/IL-8R; and 26974.5 for TNF/TNFR; all in arbitrary units) and multiplexed pool of 96 ligands (1454 for EPO/EPOR; −476 for IL-8/IL-8R; and 6485.5 for TNF/TNFR; all in arbitrary units) remains easily detectable.

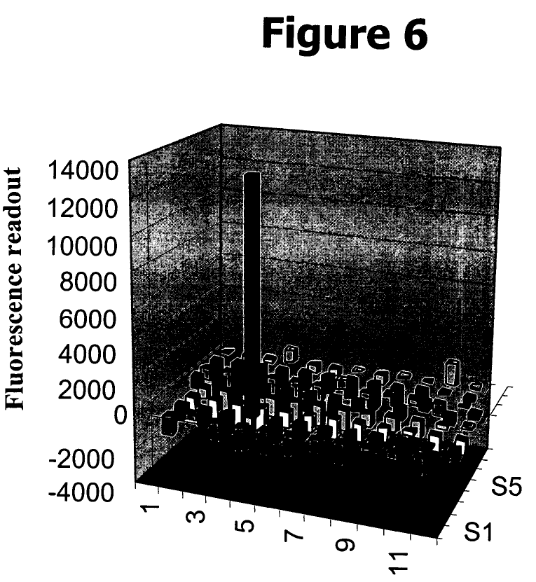

As a further demonstration of the sensitivity of this cell-based assay, FIG. 6 shows that in a 96-well plate, detection of binding of the EPO receptor from a sample having a multiplexing factor of 12 may be accomplished. Only one out of 96 wells had a fluorescence readout of 13444, which was significantly higher than the readings from the other wells.

In another variation of the invention, the cell-based assay may be used to identify an unknown polypeptide ligand. In general, as described above, a DNA construct expressing polypeptide ligand is generated by fusing DNA sequences encoding polypeptide ligand to the 5′ end of a leaderless beta-lactamase gene sequence (or to the 5′ end of other leaderless enzyme/marker (reporter) genes). The fusion gene is under the control of a promoter (FIG. 1A); the promoter may be a prokaryotic (e.g., bacterial) promoter or a eukaryotic (e.g., mammalian) promoter, or a dual expression promoter (functions both in prokaryotic system and eukaryotic system). The plasmid expressing polypeptide ligand contains a selectable marker, e.g., a gene encoding neomycin for G418 selection (Invitrogen). The DNA construct expressing receptor is generated by placing a gene encoding the receptor under the control of a eukaryotic (e.g., mammalian) promoter (FIG. 1B). The plasmid expressing receptor also contains a gene encoding neomycin for G418 selection (Invitrogen).

The plasmid expressing the receptor gene is transfected into eukaryotic (e.g., mammalian) cells. The cells stably expressing receptor are selected using G418 (Invitrogen). The plasmid expressing the ligand-lactamase fusion gene is transfected into eukaryotic (e.g., mammalian) cells or is transformed into prokaryotic (e.g., bacterial) cells. The cells transiently or stably expressing receptor contact the culture medium of cells expressing the polypeptide-ligand fusion protein. If the polypeptide ligand binds to the receptor, the polypeptide ligand-lactamase fusion protein will be located on cell surface and cannot be washed off. As seen in FIG. 2B, the bound ligand-fusion protein cleaves the lactamase substrate (e.g., CCF2 (Aurora)), and displays a change in fluorescence emission. The gene encoding the ligand for the known bound receptor is thereby identified.

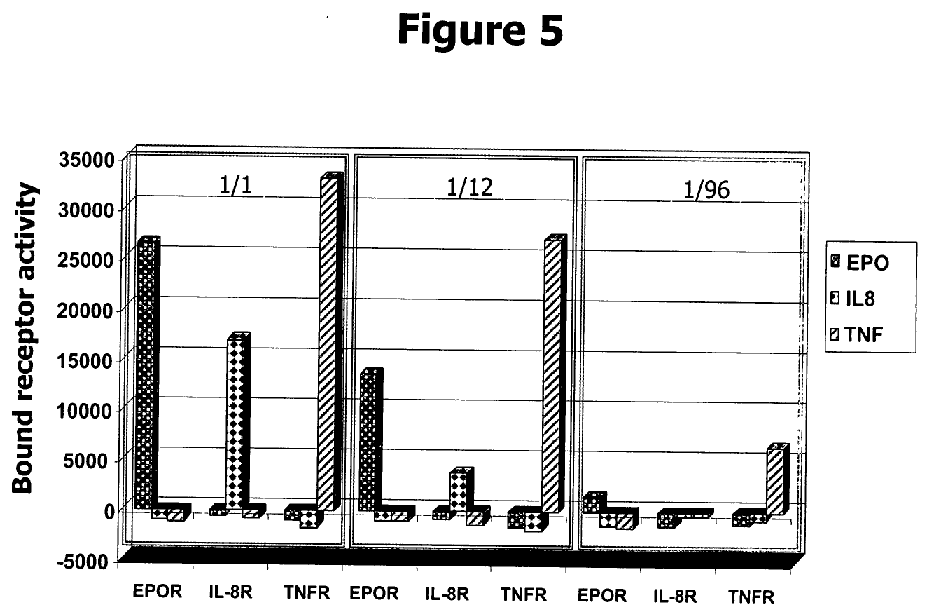

The unknown ligand may also be identified from a multiplexed pool of receptors using, e.g., a beta-lactamase reporter and CCF2 as the substrate. As shown in FIG. 5, the EPO, IL-8, and TNF receptors were placed in a pool of both 12 and 96 receptors. Compared with the fluorescent readouts (arbitrary units) for one to one binding of receptor and ligand (26398 for EPO/EPOR; 16941 for IL-9/IL-8R; and 32899 for TNF/TNFR), binding of specific receptor-ligand pairs from a multiplexed pool of 12 receptors (13570 for EPO/EPOR; 3785 for IL-8/IL-8R; and 26974.5 for TNF/TNFR; all in arbitrary units) and multiplexed pool of 96 receptors (1454 for EPO/EPOR; −476 for IL-8/IL-8R; and 6485.5 for TNF/TNFR; all in arbitrary units) remains easily detectable.

In a further variation of the invention, the cell-based assay may be used to screen for compounds, such as small molecule compounds or other reagents (such as polypeptides or nucleotides), that mediate (inhibit or enhance) the specific binding between polypeptide ligand and receptor. Generally, the plasmid expressing a polypeptide ligand reporter fusion gene and the plasmid expressing the corresponding receptor are transfected into eukaryotic (e.g., mammalian) cells independently. The cell line stably expressing ligand fusion protein and the cell line stably expressing the corresponding receptor is generated, e.g., by G148 selection (Invitrogen). The cells transiently or stably expressing polypeptide ligand and cells transiently or stably expressing receptor are cultured independently.

The cells expressing receptor contact the culture medium of cells expressing ligand fusion protein in the presence of small molecule compounds or other reagents (such as polypeptides or nucleotides). Alternatively, the cells expressing receptor can be pre-incubated with small molecule compounds or other reagents (such as polypeptides or nucleotides), then incubated with the culture medium, or the cells expressing receptor can be pre-incubated with the culture medium then incubated with small molecule compounds or other reagents (such as polypeptides or nucleotides). A sample protocol identifying small non-peptide molecules which can mediate ligand-receptor binding can be found in White, et al., J. Biol. Chem. (1998) 273: 10095-10098.

After incubation and wash, the reporter activity of polypeptide ligand bound to the cell surface is measured, and is compared with that of a control not having a small molecule compound or other reagent (such as a polypeptide or nucleotide). If small molecule compounds or other reagents bind to the receptor or to the polypeptide ligand, and mediate (inhibit or enhance) the binding between polypeptide ligand and receptor, a change (decrease or increase) in the amount of ligand fusion bound to the cell surface results, and a corresponding change (decrease or increase) in reporter activity will be detected.

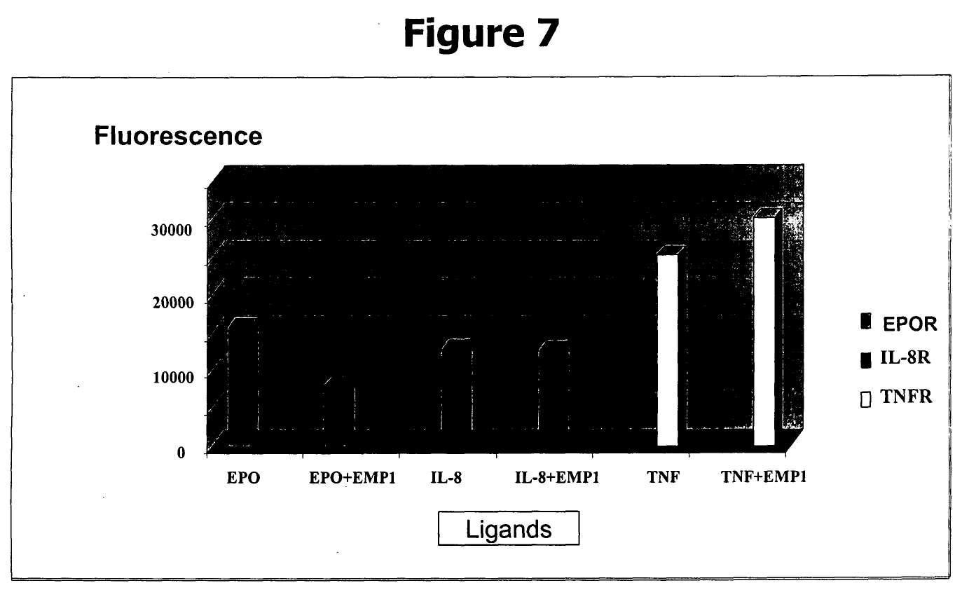

For example, as seen in FIG. 7, and further described in Example 6A, incubation of EPO-lactamase fusion proteins with EPOR in the presence of the peptide EMP1 interfered with EPO binding to EPOR. A description and use of the EMP1 peptide can be found in Skelton, et al., J. Mol. Biol. (2002) 316: 1111-1125.

The ability of this cell-based assay to identify mediators of receptor-ligand binding may allow it to screen for compounds that block viral entry, specifically entry of the Human Immunodeficiency Virus (HIV). In the native infection pathway, viral entry requires interactions between viral envelope proteins and cellular receptors.

As further described in Example 6B, a viral envelope protein can be expressed as a fusion protein ligand (i.e., coupled to a reporter such as beta-lactamase) or on a host cell membrane while the cellular receptors can also be expressed on a host cell membrane or as a fusion protein ligand coupled to a reporter (such as beta-lactamase), respectively. The binding between the cellular receptor and viral envelope protein can be detected by detecting the presence of the reporter molecule. The biological activity of small molecule compounds or other reagents (such as polypeptides or nucleotides) to block the binding between a viral envelope protein and cell receptor can then be identified through screening a large library of test compounds very efficiently.

### EXAMPLES

The following examples serve to more fully describe the manner of using the invention herein described. It is understood that these examples in no way serve to limit the scope of the invention, but rather are presented for illustrative purposes.

### Example 1

Preparation of Constructs

Constructs expressing the fusion genes of EPO (erythropoietin), IL-8 (interleukin-8), and TNF (tumor necrosis factor) polypeptide ligands with lactamase were generated, as well as constructs expressing their receptors (EPOR, IL-8R, and TNFR, respectively). Plasmids expressing peptide ligand lactamase fusion and plasmids expressing receptors all contained a gene encoding neomycin for G418 selection.

### Example 1A

Construction of Plasmids Expressing Ligand-Lactamase Fusions

We used pBK-Scripe vector (Stratagene) as the backbone to generate constructs expressing fusions of these polypeptide ligands and lactamase, i.e., TNF-lac; IL8-lac; EPO-lac;

The cDNA fragments encoding polypeptide ligands were generated with a rtPCR method using human total RNA master panel II (Clontech) as a template. The restriction sites of EcoRI or NotI were introduced at the 5′ or 3′ end of the PCR products, respectively. The leaderless beta lactamase, in which the N-terminal end sequences encoding the signal peptide sequences (amino acid residues 1 to 25) (Sutcliffe, J. G. (1978) Proc. Natl. Acad. Sci. USA 75, 3737; Kadonaga, J. T. et. al., (1984) J. Biol. Chem. 259, 2149) have been deleted, was amplified by PCR. The restriction sites for NotI or XhoI were introduced at 5′ or 3′ end of the PCR product, respectively. To increase the flexibility of lactamase, a linker consisting of three glycine residues or other flex polypeptide linker (Shamoo, Y., Abdul-Manan, N., and Williams, K. R. (1995) Nucleic Acids Res. 23, 725) was introduced between the polypeptide ligand and beta-lactamase. The PCR fragment encoding TNF, IL1, IL8, EPA, EPO, and NGF was digested with EcoRI and NotI; and the PCR fragment encoding the leaderless beta-lactamase was digested with NotI and XhoI. The digested fragments encoding ligand proteins (EcoRI/NotI ends) were ligated with the digested fragment encoding leaderless beta-lactamase (NotI/XhoI ends). The resulting fragments encoding ligand-lactamase fusion proteins were then cloned into pBK-Scripe vector between EcoRI and XhoI. The resulting pBK-Scripe-TNF-lac, pBK-Scripe-IL1-lac, BK-CMV-IL8-lac, pBK-Scripe-EPA-lac, pBK-Scripe-EPO-lac, and pBK-Scripe-NGF-lac expression vectors were confirmed by sequencing.

### Example 1B

Construction of Plasmids Expressing Receptors

cDNA fragments encoding TNFR; IL-8R; EPOR; were generated by PCR with DNA clones containing their coding sequences as templates. The products were cloned into the pcDNA3.1DNV5-His-TOPO vector (Invitrogen). The resulting plasmids, pcDNA3.1D-TNFR, pcDNA3.1D-IL8R, pcDNA3.1D-EPOR, were confirmed by sequencing.

### Example 1C

Confirmation of Expression and Secretion of Ligand Lactamase Fusion

Plasmids expressing polypeptide ligand lactamase fusions were introduced into eukaryotic (e.g., mammalian) cell lines or prokaryotic (e.g., bacterial) cell lines. For example, the plasmids expressing ligand fusions were transfected into eukaryotic (e.g., mammalian) cells, such as the 293-cell line, CHO cell line, COS cell line, or HeLa cell line, with lipofectamine 2000 reagent (Invitrogen). After growth, the culture of cells transfected with plasmids expressing ligand fusion was collected to test lactamase activity with a nitrocefin color assay (Calbiochem). If the polypeptide ligand lactamase fusion is expressed and secreted out of the cells, the culture medium will show a significantly higher lactamase activity comparing with the controls transfected with plasmid expressing leaderless lactamase (Moore, et al. (1997) Annal. Biochem. 247, 203).

### Example 1D

Confirmation of the Expression of Receptor

Plasmids expressing receptors were transfected into eukaryotic (e.g., mammalian) cell lines. For example, the plasmids expressing receptors were transfected into mammalian cell lines, such as the 293-cell line, CHO cell line, COS cell line, or HeLa cell line, with lipofectamine 2000 reagent (Invitrogen). The cell line stably expressing receptor was selected with G418 (Invitrogen). The transfected cell line or selected stable cells are propagated and collected for SDS polyacrylamide gel electrophoresis and western analysis with specific antibody or an anti-V5 antibody (Invitrogen) to confirm the expression of receptor.

### Example 2

Selecting Natural or Recombinant Polypeptide Ligands for Specific Receptors

Preparation of cDNA or Combinatorial Oligo Nucleotides

Total RNA was isolated from organs, tissues or cells using TRIZOL (Life Technologies). mRNA was selected using Oligotex beads (Qiagen). cDNAs encoding natural polypeptides were prepared using random primers and Superscript II as in instruction manual (Invitrogen). The resulting cDNAs contained a 5′ EcoRI end and a 3′ NotI end. Combinatorial oligo nucleotides encoding recombinant polypeptides were generated as described (Tan, R. and Frankel, A.D. (1998) Proc. Natl. Acad. Sci. USA 94, 11887). The resulting combinatorial oligo nucleotides contained a 5′ EcoRI end and a 3′ NotI end.

Libraries

In order to improve the efficiency of library construction, we used pBK-Scripe-TNF-lac vector as the cloning vector to construct a library expressing natural or recombinant polypeptide lactamase fusions. pBK-Scripe-TNF-lac vector was digested with EcoRI and NotI to remove the DNA fragment encoding TNF, the resulting pBK-Scripe-lac(EcoRI/NotI cut) was ligated with cDNAs encoding natural polypeptides or combinatorial oligo nucleotides encoding recombinant polypeptides. The ligation products were transformed into DH-10B electron competent cells by electroplation (Invitrogen). The resulting colonies were picked to grow in multiple-well plates.

Construction of a Cell Line Stably Expressing Receptor

Plasmids expressing receptor were generated as described above.

A cell line stably expressing receptor was generated and confirmed also as described above.

Identifying the Clones Expressing Secreted Polypeptide Lactamase Fusion

The plasmid DNAs were prepared with Quick kit as described (Qiagen), and transfected into a eukaryotic (e.g. mammalian) cell line with lipofectamin 2000 reagent (Invitrogen) in multiple-well format. After growth, the culture medium of each well was collected to test lactamase activity to identify the expression and secretion of polypeptide lactamase fusion by nitrocefin assay (Calbiochem). If the culture medium showed lactamase activity, then the corresponding clones were known to have expressed the polypeptide ligand lactamase fusion.

Screening the Library Expressing Natural or Recombinant Polypeptide Ligands against the Cell Line Expressing Specific Receptor.

The identified clones expressing secreted polypeptide lactamase fusion were chosen to screen polypeptide ligands for a specific receptor. The plasmid DNAs were transfected into a eukaryotic (e.g. mammalian) cell line with lipofectamine 2000 reagents (Invitrogen) in multiple-well format. The cells transiently or stably expressing special receptor were cultured in multiple-well plates. After growth, the culture medium of the cells expressing receptor in each well was removed, and the culture medium of the cells expressing secreted polypeptide ligand fusion was added to each well. To decrease the work amount of screening, we used the sib-screen method as described (Chen, H. and Leder, P. (1999) Nucleic Acids Research, 27, 1219). After the mixture was incubated at 4° C. for 1 hour, the culture medium was removed. The cells expressing receptor were washed three times with PBS containing 1% FBS (Invitrogen). A lactamase substrate, such as CCF2 in 100 μl volume of 1 μl, was added to each well, and incubated. The fluorescence emission at 447 nm with excitation at 409 nm was measured using Spectra Max Gemim (Molecular Devices) in each well at different time points. The higher read out of fluorescence emission at 447 nm represented the higher lactamase activity of the binding complex of ligand fusion and receptor, and indicated the higher binding affinity of the polypeptide ligand to the receptor than the binding of the reference ligand to the same receptor.

### Example 3

Selecting Receptors for Specific Polypeptide Ligands

Preparation of cDNA Expression Library

Total RNA was isolated from organs, tissues or cells using TRIZOL (Life Technologies). To enrich cDNA library for membrane proteins, polysomal mRNA enriched in encoding membrane proteins or secreted proteins was prepared from membrane-bound RNA as described (Diehn, M. et al., (2000) Nature Genetics 25, 58). mRNA was selected using Oligotex beads (Qiagen) from total RNA or membrane-bound polysome RNA. cDNAs were prepared using oligo dT primer and Superscript II as described in the instruction manual (SUPERSCRIPT™ Plasmid System for cDNA Synthesis and Plasmid Cloning (GIBCOBRL, CAT.NO 18248-013)). The resulting cDNAs were ligated into a eukaryotic (e.g., mammalian) expression vector (SUPERSCRIPT™ Plasmid System for cDNA Synthesis and Plasmid Cloning (GIBCOBRL, CAT.NO 18248-013)). The ligation products were transformed into DH-10B electron competent cells by electroplation (Invitrogen). The resulting colonies were picked to grow in multiple-well plates.

Construction of Plasmid Expressing Specific Polypeptide Ligand-Lactamase Fusion Protein

We used the pBK-Scripe vector (Stratagene) as the backbone to generate a construct expressing fusion of specific polypeptide ligand and lactamase.

The cDNA fragment encoding specific polypeptide ligand was generated with a rtPCR method using total RNA as a template. The restriction sites of EcoRI or NotI were introduced at the 5′ or 3′ end of the PCR products, respectively. The leaderless beta lactamase, in which the N-terminal end sequences encoding the signal peptide sequences (amino acid residues 1 to 25) (Sutcliffe, J. G. (1978) Proc. Natl. Acad. Sci. USA 75, 3737; Kadonaga, J. T. et. al., (1984) J. Biol. Chem. 259, 2149) have been deleted, was amplified by PCR. The restriction sites for NotI or XhoI were introduced at 5′ or 3′ end of the PCR product, respectively. To increase the flexibility of lactamase, a linker consisting of three glycine residues or other flex polypeptide linker (Shamoo, Y., Abdul-Manan, N., and Williams, K. R. (1995) Nucleic Acids Res. 23, 725) was introduced between the polypeptide ligand and beta-lactamase. The PCR fragment encoding specific polypeptide ligand was digested with EcoRI and NotI; and the PCR fragment encoding the leaderless beta-lactamase was digested with NotI and XhoI. The digested fragment encoding polypeptide ligand (EcoRI/NotI ends) was ligated with the digested fragment encoding leader-less beta-lactamase (NotI/XhoI ends). The resulting fragment encoding specific polypeptide ligand-lactamase fusion was ligated into pBK-Scripe vector between EcoRI and XhoI. The resulting vector expressing specific polypeptide ligand lactamase fusion was confirmed by sequencing.

Identification of Receptor for Specific Ligand

The plasmid DNAs were prepared with Quick kit as described (Qiagen), and transfected into a eukaryotic (e.g. mammalian) cell line with lipofectamin 2000 reagent (Invitrogen) in multiple-well format. The transfected cells were cultured in multiple-well plates. To decrease the work amount of screening, we used the sib-screen method as described (Chen, H. and Leder, P. (1999) Nucleic Acids Research, 27, 1219). After growth, the culture medium of the cells transfected with plasmids DNAs of cDNA library in each well was removed, and the culture medium of the cells expressing specific polypeptide ligand fusion was added to each well. After the mixture was incubated at 4° C. for 2 hours, the culture medium was removed. The cells transfected with plasmids DNAs of cDNA library were washed three times with PBS containing 1% FBS (Invitrogen). A lactamase substrate, such as CCF2 in 100 μl volume of 1 μm, was added to each well, and incubated. The fluorescence emission at 447 nm with excitation at 409 nm was measured using Spectra Max Gemim (Molecular Devices) in each well at different time points.

When cells in some well(s) were transfected with plasmids expressing receptor for the specific polypeptide ligand, the expressed receptor became located on their membranes. The polypeptide ligand bound to the surface of the cells in that (or those) well(s), was not washed off. The ligand fusion bound on cell surface cleaved the lactamase substrate (e.g., CCF2 (Aurora)), and displayed a change in fluorescence emission in some well(s). The plasmids transfected into that (or those) well(s) were then sequenced. The gene encoding receptor for the specific ligand was then identified.

### Example 4

Receptor-Ligand Binding

Cells transfected with plasmids expressing a ligand lactamase fusion and cells transiently or stably expressing receptor were independently cultured in multiple well (e.g., 96-well, 384-well, or 1536-well) plates. After growth, the culture medium of the cells transiently or stably expressing receptor was removed, and the culture medium of the cells expressing ligand lactamase fusion was added to the wells of cells stably expressing receptor. After the mixture was incubated at 4° C. for 1 hour, the medium was removed. The cells stably expressing receptor were washed three times with PBS containing 1% FBS (Invitrogen), and a lactamase substrate, such as CCF2 in 100 μl volume of 1 μl, was added to the washed cells in each well, and incubated. The fluorescence emission at 447 nm with excitation at 409 nm was measured using the Spectra Max Gemim (Molecular Devices) at different time points. The increase of fluorescence emission at 447 nm represented the lactamase activity of ligand fusion bound on the cell surface.

### Example 4A

Detecting Binding of EPO Receptor to EPO Ligand

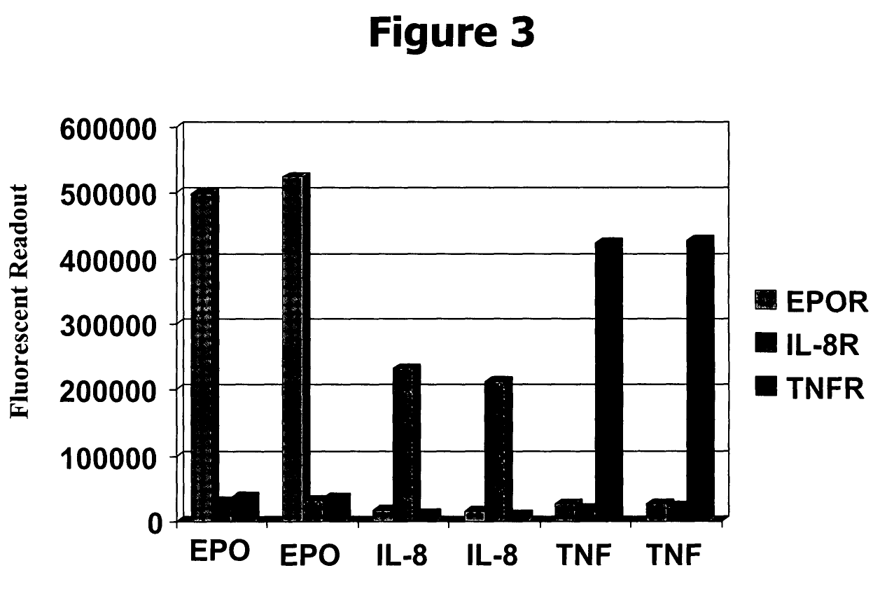

The EPO receptor and EPO, IL-8, and TNF ligand lactamase fusion proteins were expressed as described above. The EPO, IL-8, and TNF ligand lactamase fusions were contacted with the EPO receptor in duplicate experiments. As shown in FIG. 3, only binding of EPO ligand with EPO receptor resulted in a high fluorescence readout, as compared to the readouts for EPO ligand combined with the IL-8 receptor and with the TNF receptor.

### Example 4B

Detecting Binding of IL-8 Receptor to IL-8 Ligand

The IL-8 receptor and IL-8, EPO, and TNF ligand lactamase fusion proteins were expressed as described above. The IL-8, EPO, and TNF ligand lactamase fusions were contacted with the IL-8 receptor in duplicate experiments. As shown in FIG. 3, only binding of IL-8 ligand with IL-8 receptor resulted in a high fluorescence readout, as compared to the readouts for IL-8 ligand combined with the EPO receptor and with the TNF receptor.

### Example 4C

Detecting Binding of TNF Receptor to TNF Ligand

The TNF receptor and TNF, EPO, and IL-8 ligand lactamase fusion proteins were expressed as described above. The TNF, EPO, and IL-8 ligand lactamase fusions were contacted with the TNF receptor in duplicate experiments. As shown in FIG. 3, only binding of TNF ligand with TNF receptor resulted in a high fluorescence readout, as compared to the readouts for TNF ligand combined with the EPO receptor and with the IL-8 receptor.

### Example 5

Receptor-Ligand Binding Using a Multiplexed Pool of Ligand-Reporter Fusion Proteins

As described above, receptor-ligand binding may be detected from a pool of ligands or receptors having a multiplexing factor of at least about.

### Example 5A

Detecting EPO Receptor From a Multiplexed Pool of Ligands

EPO, IL-8, and TNF receptors and ligands were expressed as described above. Known ligands, including the EPO ligand, were grouped to form a multiplexed pool of 12 ligands and a multiplexed pool of 96 ligands. Compared to the fluorescence readout for one on one receptor-ligand binding for EPO/EPOR (26398 arbitrary units), the fluorescence readout (and thus, binding) was less for EPO/IL-8R (−503) and EPO/TNFR (−1027). From a multiplexed pool of both 12 and 96 ligands, the fluorescence readout for EPO/EPOR binding continued to be significantly higher than that for EPO/IL-8R (13570 (12 ligands) and 1454 (96 ligands) for EPO/EPOR vs. −785 (12 ligands) and −1334 (96 ligands) for EPO/IL-8R; all in arbitrary units); and EPO/TNFR (13570 (12 ligands) and 1454 (96 ligands) for EPO/EPOR vs. −1598 (12 ligands) and −1177 (96 ligands) for EPO/TNFR).

### Example 5B

Detecting IL-8 Receptor From a Multiplexed Pool of Ligands

EPO, IL-8, and TNF receptors and ligands were expressed as described above. Known ligands, including the IL-8 ligand, were grouped to form a multiplexed pool of 12 ligands and a multiplexed pool of 96 ligands. Compared to the fluorescence readout for one on one receptor-ligand binding for IL-8/IL-8R (16941 arbitrary units, the fluorescence readout (and thus, binding) was less for IL-8/EPOR (−902) and IL-8/TNFR (−1677). From a multiplexed pool of both 12 and 96 ligands, the fluorescence readout for IL-8/IL-8R continued to be significantly higher than that for IL-8/EPOR (3785 (12 ligands) and −476 (96 ligands) for IL-8/IL-8R vs. −1059 (12 ligands) and: 1448 (96 ligands) for IL-8/EPOR) and IL-8/TNFR (3785 (12 ligands) and −476 (96 ligands) for IL-8/IL-8R vs. −147 (12 ligands) and −718 (96 ligands) for IL-8/TNFR).

### Example 5C

Detecting TNF Receptor From a Multiplexed Pool of Ligands

EPO, IL-8, and TNF receptors and ligands were expressed as described above. Known ligands, including the TNF ligand, were grouped to form a multiplexed pool of 12 ligands and a multiplexed pool of 96 ligands. Compared to the fluorescence readout for one on one receptor-ligand binding for TNF/TNFR (32899 arbitrary units), the fluorescence readout (and thus, binding) was less for TNF/EPOR (−1113.5) and TNF/IL-8R (−814.5). From a multiplexed pool of both 12 and 96 ligands, the fluorescence readout for TNF/TNFR continued to be significantly higher than that for TNF/EPOR (26974.5 (12 ligands) and 6485.5 (96 ligands) for TNF/TNFR vs. −1005.5 (12 ligands) and −1634 (96 ligands) for TNF/EPOR) and TNF/IL-8R (26974.5 (12 ligands) and 6485.5 (96 ligands) for TNF/TNFR vs. −1275.5 (12 ligands) and −381 (96 ligands) for TNF/IL-8R).

### Example 5D

Detecting EPO Ligand From a Multiplexed Pool of Receptors

EPO, IL-8, and TNF receptors and ligands were expressed as described above. Known receptors, including the EPO receptor, were grouped to form a multiplexed pool of 12 receptors and a multiplexed pool of 96 receptors. Compared to the fluorescence readout for one on one receptor-ligand binding for EPOR/EPO (26398 arbitrary units), the fluorescence readout (and thus, binding) was less for EPOR/IL-8 (−902) and EPOR/TNF (−1113.5). From a multiplexed pool of both 12 and 96 receptors, the fluorescence readout for EPOR/EPO continued to be significantly higher than that for EPOR/IL-8 (13570 (12 ligands) and 1454 (96 ligands) for EPOR/EPO vs. −1059 (12 ligands) and −1448 (96 ligands) for EPOR/IL-8) and EPOR/TNF (13570 (12 ligands) and 1454 (96 ligands) for EPOR/EPO vs. −10005.5 (12 ligands) and −1634 (96 ligands) for EPOR/TNF).

### Example 5E

Detecting IL-8 Ligand From a Multiplexed Pool of Receptors

EPO, IL-8, and TNF receptors and ligands were expressed as described above. Known receptors, including the IL-8 receptor, were grouped to form a multiplexed pool of 12 receptors and a multiplexed pool of 96 receptors. Compared to the fluorescence readout for one on one receptor-ligand binding for IL-8R/IL-8 (16941 arbitrary units, the fluorescence readout (and thus, binding) was less for IL-8R/EPO (−503) and IL-8R/TNF (−814). From a multiplexed pool of both 12 and 96 receptors, the fluorescence readout for IL-8R/IL-8 continued to be significantly higher than that for the IL-8R/EPO (3785 (12 ligands) and −476 (96 ligands) for IL-8R/IL-8 vs. −785 (12 ligands) and −1334 (96 ligands) for IL-8R/EPO) and IL-8R/TNF(3785 (12 ligands) and −476 (96 ligands) for IL-8R/IL vs. −1275.5 (12 ligands) and −381 (96 ligands) for IL-8R/TNF).

### Example 5F

Detecting TNF Ligand From a Multiplexed Pool Receptors

EPO, IL-8, and TNF receptors and ligands were expressed as described above. Known receptors, including the TNF receptor, were grouped to form a multiplexed pool of 12 receptors and a multiplexed pool of 96 receptors. Compared to the fluorescence readout for one on one receptor-ligand binding for TNFR/TNF (32899 arbitrary units), the fluorescence readout (and thus, binding) was less for TNFR/EPO (−1027) and TNFR/IL-8 (−1677). From a multiplexed pool of both 12 and 96 receptors, the fluorescence readout for TNFR/TNF continued to be significantly higher than that for the TNFR/EPO (26974.5 (12 ligands) and 6485.5 (96 ligands) for TNFR/TNF vs. −1598 (12 ligands) and −1177 (96 ligands) for TNFR/EPO) and TNFR/IL-8 (26974.5 (12 ligands) and 6485.5 (96 ligands) for TNFR/TNF vs. −1847 (12 ligands) and −718 (96 ligands) for TNFR/IL-8).

### Example 6

Mediation of Receptor-Ligand Binding

After the specific pairing of polypeptide ligand and receptor has been identified as described above, we can screen for small molecule compounds or other reagents (such as polypeptides or nucleotides) that mediate their specific binding.

Cells transiently or stably expressing fusion protein of polypeptide ligand and lactamase and cells transiently or stably expressing receptor were independently cultured in multiple-well (96-well, 384-well, or 1536-well) plates. After growth, the culture medium of the cells expressing receptor was removed, and the culture medium of the cells expressing the ligand lactamase fusion and small molecule compounds or other reagents (such as polypeptides or nucleotides) were added to each well. After the mixture was incubated at 4° C. for 1 hour, the culture medium was removed.

Alternatively, the cells expressing receptor were pre-incubated with small molecule compounds or other reagents (such as polypeptides or nucleotides) then incubated with the culture medium, or the cells expressing receptor were pre-incubated with the culture medium then incubated with small molecule compounds or other reagents (such as polypeptides or nucleotides). The cells expressing receptor were washed three times with PBS containing 1% FBS (Invitrogen).

A lactamase substrate, such as CCF2 in 100 μl volume of 100 μl, was added to the washed cells in each well, and incubated. The fluorescence emission at 447 nm with excitation at 409 nm was measured using Spectra Max Gemim (Molecular Devices) at different time points, and compared with that of a control not including a small molecule compound. If the small molecule compounds or other reagents (such as polypeptides or nucleotides) had bound to the receptor or to the polypeptide ligand, and mediated (inhibited or enhanced) the binding between polypeptide ligand and receptor, a change (increase or decrease) in the amount ligand fusion bound on the cell surface would result, and thus display a change (decrease or increase) in read out of lactamase activity.

### Example 6A

Mediation of Specific Binding Between EPO Ligand and EPO Receptor

EPO ligand lactamase fusions and receptor were expressed from 293-cells as described above. After culture for 48 hours, cells expressing the EPO receptor were incubated with the culture medium from cells expressing the EPO lactamase fusion proteins in the presence of 100 μl of EMP1 peptide. After incubation, the medium was removed and the cells washed three times with buffer. CCF2 substrate was added to the cells and fluorescence emission measured, also as described above. As seen in FIG. 7, the EMP 1 peptide specifically interfered with EPO ligand-receptor binding, not IL-8 or TNF ligand-receptor binding.

### Example 6B

Mediation of Viral Entry

The viral envelope protein can either be expressed as a fusion protein ligand with beta-lactamase or on the cell surface as a membrane protein. Vice versa, the cellular viral receptor protein can be expressed on the cell surface or as a fusion protein ligand with beta-lactamase. For example, human immunodeficiency virus (HIV) can be used.

The viral entry of HIV depends on the presence of cell surface molecular CD4 and a viral co-receptor. The binding between viral gp120 and gp41 to cell receptor CD4 and co-receptor results in viral entry and viral infection. Mechanisms to block the viral entry can potentially block the viral infection and result in a cure of acquired immunodeficiency syndrome (AIDS).

Using our cell-based assay, CD4 molecules may be expressed as a fusion protein ligand coupled with beta-lactamase while the viral glycoprotein, gp120 and gp41 may be expressed on a host cell membrane. Small molecule compounds or other reagents (such as polypeptides or nucleotides) can then be added to the incubation mixture containing CD4 fusion molecules and host cells expressing viral glycoproteins. Alternatively, the host cells expressing viral glycoprotein are pre-incubated with small molecule compounds or other reagents (such as polypeptides or nucleotides) then incubated with the culture medium containing CD4 fusion molecules. Or, the host cells expressing viral glycoprotein may be pre-incubated with the culture medium containing CD4 fusion molecules then incubated with small molecule compounds or other reagents (such as polypeptides or nucleotides). The specific small molecule compounds or other reagents (such as polypeptides or nucleotides) that blocks the interaction between CD4 and viral glycoprotein can be identified as described in Example 4A.

The following references are incorporated herein in their entirety.

1. Moore, J. T., Davis, S. T., and Dev, I. K. (1997) Annal. Biochem. 247, 203.

2. Chubb, A. J. et al. (1998) Microbiology 144, 1619.

3. Chen, H. and Leder, P. (1999) Nucleic Acids Research, 27, 1219.

4. Zlokarnik, G. et al (1998) Science 279, 84.

5. Raz, E. et al. (1998) Development Biology 203 290.

6. Smith, C. A. et. al., (1990) Science 248, 1019.

7. Vigers, G. P. et al. (1997) Nature 386, 190.

8. Lee, J. et al. (1992) J. Biol. Chem. 267, 16283.

9. Himanen, J -P. et. al., (2001) Nature 414, 933.

10. D'Andrea. A. D., Lodish, H. F., and Wong, G. G. (1989) Cell 57, 277.

11. Hirashima, N., Nisho, M., and Nakanishi, M. (2000) Bio. Pharm. Bull. 23, 1097.

12. Sutcliffe, J. G. (1978) Proc. Natl. Acad. Sci. USA 75, 3737.

13. Kadonaga, J. T. et. al., (1984) J. Biol. Chem. 259, 2149.

14. Shamoo, Y., Abdul-Manan, N., and Williams, K. R. (1995) Nucleic Acids Res. 23, 725.

15. Tan, R. and Frankel, A. D. (1998) Proc. Natl. Acad. Sci. USA 94, 11887.

All publications, patents, and patent applications cited herein are hereby incorporated by reference in their entirety for all purposes to the same extent as if each individual publication, patent, or patent application were specifically and individually indicated to be so incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit and scope of the appended claims.

What is claimed is:

1. A method of identifying, in a sample, a receptor which is capable of binding to a known ligand, comprising the steps of:
a. providing a fusion molecule comprising said known ligand covalently linked to a threshold reporter enzyme molecule, said threshold reporter enzyme molecule being capable of reacting with a suitable substrate so as to generate a detection signal,
b. contacting said sample containing said receptor with said fusion molecule to form a complex between said receptor and said known ligand, and
c. detecting the presence of said complex by incubating said complex with said suitable substrate so as to generate the detection signal, thereby identifying the receptor.

2. The method of claim 1, wherein step (b) further comprises washing said sample such that any fusion molecule not forming a complex with said receptor is removed.

3. The method of claim 1 wherein said method further comprises a cell-based assay.

4. The method of claim 1, wherein the sample comprises mammalian cells expressing said receptor.

5. The method of claim 1, wherein the detection signal is selected from the group consisting of a fluorescence signal, a chemiluminescent signal, and a calorimetric signal.

6. The method of claim 1, wherein said ligand is a polypeptide ligand.

7. The method of claim 1, wherein said ligand is soluble.

8. The method of claim 1, wherein said ligand is insoluble.

9. The method of claim 1, wherein said ligand is a naturally occurring polypeptide molecule.

10. The method of claim 1, wherein said ligand is a recombinant polypeptide molecule.

11. The method of claim 1, wherein the receptor is a cell surface receptor.

12. The method of claim 1, wherein said threshold reporter enzyme molecule is beta-Lactamase.

13. The method of claim 12, wherein said suitable substrate is a beta-Lactamase substrate.

14. The method of claim 13, wherein said beta-Lactamase substrate is CCF2.

15. The method of claim 13, wherein said beta-Lactamase substrate is nitrocefin.

16. A method of identifying a ligand from a plurality of polypeptide molecules in a sample, said ligand being capable of binding to a known receptor, comprising the steps of:
a. providing a fusion molecule comprising said ligand covalently linked to a threshold reporter enzyme molecule, said threshold reporter enzyme molecule being capable of reacting with a suitable substrate to generate a detection signal,
b. contacting said sample containing said known receptor with said fusion molecule to form a complex between said known receptor and said ligand, and
c. detecting the presence of said complex by incubating said complex with said suitable substrate so as to generate the detection signal, thereby identifying the ligand.

17. The method of claim 16, wherein step (b) further comprises washing said sample such that any fusion molecule not forming the complex with said receptor is removed.

18. The method of claim 16 wherein said method further comprises a cell-based assay system.

19. The method of claim 16, wherein the sample comprises mammalian cells expressing said receptor.

20. The method of claim 16, wherein the detection signal is selected from the group consisting of a fluorescence signal, a chemiluminescent signal, and a colorimetric signal.

21. The method of claim 16, wherein said ligand is a polypeptide ligand.

22. The method of claim 16, wherein said ligand is soluble.

23. The method of claim 16, wherein said ligand is insoluble.

24. The method of claim 16, wherein said ligand is a naturally occurring polypeptide molecule.

25. The method of claim 16, wherein said ligand is a recombinant polypeptide molecule.

26. The method of claim 16, wherein the receptor is a cell surface receptor.

27. The method of claim 16, wherein said threshold reporter enzyme molecule is beta-Lactamase.

28. The method of claim 27, wherein said suitable substrate is a beta-Lactamase substrate.

29. The method of claim 28, wherein said beta-Lactamase substrate is CCF2.

30. The method of claim 28, wherein said beta-Lactamase substrate is nitrocefin.

31. A method of identifying a compound that mediates the binding activity between a known ligand and a known receptor, comprising the steps of:
a. providing a fusion molecule comprising said known ligand covalently linked to a threshold reporter enzyme molecule, said threshold reporter enzyme molecule being capable of reacting with a suitable substrate so as to generate a detection signal,
b. contacting said fusion molecule with said known receptor in the presence of said compound, so as to form a complex between said known receptor and said known ligand,
c. detecting the presence of said complex by incubating said complex with said suitable substrate so as to generate the detection signal, and
d. determining the amount of the detection signal and comparing that amount to an amount of a detection signal obtained in the absence of said compound, wherein a reduction in the amount of the detection signal in the presence of said compound indicates that said compound inhibits the binding of said ligand to said receptor, and a increase in the amount of the detection signal in the presence of said compounds indicates that said compound enhances the binding of said ligand to said receptor.

32. The method of claim 31, wherein in step (b), said fusion molecule is contacted with said known receptor first, followed by contacting with said compound.

33. The method of claim 31, wherein in step (b), said fusion molecule is contacted with said compound first, followed by contacting with said known receptor.

34. The method of claim 31, wherein in step (b), said known receptor is contacted with said compound first, followed by contacting with said fusion molecule.

35. The method of claim 31, wherein an increase in said detection signal in step (d) indicates that said compound in an enhancer of the binding between said known ligand and said known receptor.

36. The method of claim 31, wherein a decrease in said detection signal in step (d) indicates that said compound is an inhibitor of the binding between said known ligand and said known receptor.

37. The method of claim 31, wherein step (b) further comprises washing said sample such that any fusion molecule not forming the complex with said receptor is removed.

38. The method of claim 31 wherein said method further comprises a cell-based assay.

39. The method of claim 31, wherein the detection signal is selected from the group consisting of a fluorescence signal, a chemiluminescent signal, and a colorimetric signal.

40. The method of claim 31, wherein said ligand is a polypeptide ligand.

41. The method of claim 31, wherein said ligand is soluble.

42. The method of claim 31, wherein said ligand is insoluble.

43. The method of claim 31, wherein said ligand is a naturally occurring polypeptide molecule.

44. The method of claim 31, wherein said ligand is a recombinant polypeptide molecule.

45. The method of claim 31, wherein said receptor is a cell surface receptor.

46. The method of claim 31, wherein said ligand is the tumor necrosis factor alpha, and said receptor is the tumor necrosis factor receptor 2.

47. The method of claim 31, wherein said ligand is the interleukin-8, and said receptor is the interleukin-8 receptor A.

48. The method of claim 31, wherein said ligand is erythropoietin, and said receptor is the erythropoietin receptor.

49. The method of claim 31, wherein said threshold reporter enzyme molecule is beta-Lactamase.

50. The method of claim 49, wherein said suitable substrate is a beta-Lactamase substrate.

51. The method of claim 50, wherein said beta-Lactamase substrate is CCF2.

52. The method of claim 50, wherein said beta-Lactamase substrate is nitrocefin.

53. The method of claim 31, wherein said compound is an organic, or inorganic chemical molecule.

54. The method of claim 31, wherein said compound is a small peptide molecule.

55. The method of claim 31, wherein said known ligand is a viral envelope protein.

56. The method of claim 31, wherein said known receptor is a cellular viral receptor protein.

57. A method of identifying for a compound that blocks viral entry and inhibits viral infection comprising the method of claim 31 wherein said known ligand is a viral envelope protein, and said known receptor is a cellular viral receptor protein.

58. A method of identifying for a compound that blocks viral entry and inhibits viral infection comprising the method of claim 31 wherein said known ligand is a cellular viral receptor protein, and said known receptor is viral envelope protein.

59. The method of claim 57, wherein said known ligand is selected from the group consisting of viral glycoprotein, gp120, and gp41.

60. The method of claim 57, wherein said known receptor is a CD4 molecule.

61. The method of claim 58, wherein said known ligand is a CD4 molecule.

62. The method of claim 58, wherein said known receptor is selected from the group consisting of viral glycoprotein, gp120, and gp41.

63. A composition for use in identifying specific binding activity between a ligand and a receptor, comprising a ligand covalently linked to a threshold reporter enzyme molecule, said threshold reporter enzyme molecule being capable of reacting with a suitable substrate so as to generate a detection signal.

64. A composition for use in identifying a compound which interfers with the binding of a known receptor to a known ligand using the method of claim 31, comprising said compound, said known receptor, and said known ligand covalently linked to a threshold reporter enzyme molecule, said threshold reporter enzyme molecule being capable of reacting with a suitable substrate so as to generate a detection signal.
